(19) United States
(12) Patent Application Publication
(10) Pub. No.: US 2007/0078477 A1
(43) Pub. Date: Apr. 5, 2007

(54) Anatomical spacer and method to deploy

(76) Inventors: Scott H. Heneveld SR., Whitmore, CA (US); James C. Thomas JR., Las Vegas, NV (US); Donald Fareed, Santa Barbara, CA (US)

Correspondence Address: CHRISTIE, PARKER & HALE, LLP, PO BOX 7068, PASADENA, CA, 91109-7068, US

(21) Appl. No.: 11/348,740

(22) Filed: Feb. 6, 2006

Related U.S. Application Data
(60) Provisional application No. 60/649,689, filed on Feb. 4, 2005.

(51) Int. Cl. A61M 29/00

(52) U.S. Cl. 606/191

(57) ABSTRACT

An anatomical spacer is provided which includes a bladder expandable from a deflated position to an inflated position. A membrane extends between opposing surfaces of the bladder, and an inflation port is placed in communication with the bladder. The inflation port is configured to receive inflation media to fill the bladder from the deflated position to the inflated position. A method is also provided to deploy the spacer into an anatomical location from a cannula.

### CROSS-REFERENCE TO RELATED APPLICATION(S)

The U.S. patent application claims the priority benefit of U.S. Provisional Patent Application No. 60/649,689, filed Feb. 4, 2005, the entire content of which is incorporated herein.

### FIELD OF THE INVENTION

The present invention relates to expandable implants for the treatment of anatomic disorders, including, but not limited to joint disorders, neurological disorders, or infections, and methods and apparatuses for delivering and removing the same.

### BACKGROUND OF THE INVENTION

Bursitis is inflammation of a bursa, a small sac of fluid that cushions and lubricates an area between tendon and bone or around a joint. The inflammation can cause the bursa to swell with fluid. Bursitis can occur anywhere in the body where there is a bursa, usually near a joint. The condition is often painful.

A tendon is the end part of a muscle that attaches the muscle to the bone. The normally very elastic and soft muscle tapers off at the end to form the dense and stiff tendon. While this density makes the tendon stronger, the lack of elasticity of the tendon and the constant pulling on its attachment to the bone with movement, makes it much more susceptible to a low level of tearing at a microscopic level. This tearing will produce the inflammation and irritation known as tendonitis.

Rotator cuff disorders are irritations in, or damage to, tendons around the shoulder. These disorders include inflammation of the tendons (tendonitis) or the bursa (bursitis), a calcium buildup in the tendons, or partial or complete tears of the tendon. The rotator cuff is a group of tendons and their related muscles that helps keep the upper arm bone securely placed, or seated, into the socket of the shoulder blade. Rotator cuff disorders are usually caused by a combination of factors, such as normal wear and tear. Age-related degeneration slowly damages the rotator cuff, causing one or more tendons to rub against the bones (impingement). Underneath the acromion (subacromial space) is a bursa, which will get inflamed with shoulder impingement and often becomes the cause of a chronic irritation, decreased range of motion, and loss of strength. Compression caused by bursitis is detrimental to the healing process. If non-surgical treatment has failed to relieve impingement, the abnormally swollen and inflamed bursa is often removed. This involves an invasive and expensive open surgical decompression procedure.

In addition to the problems that exist with the rotator cuff, the same issues appear with bursitis and tendonitis in other joints of the body. The ability to avoid open surgery for treating these conditions and relieve impingement is desirable for effective repair of joint disorders.

Accordingly, there exists a need for devices and methods for treating joint disorders that overcome the problems and inadequacies of treatments currently available. Particularly, there is a need for percutaneous modality to deliver implants that effectively provide decompression and relieve the adverse effects of joint disorders. The same need is present for other anatomical related conditions, such as the isolation and treatment of tumors.

### SUMMARY OF THE INVENTION

The present invention relates to temporary and permanent expandable implants for the mechanical creation and maintenance of spaces in anatomical locations, and methods and apparatuses for delivering the same. The implants generally comprise a compressed form having a size adapted for insertion via a cannula into the prescribed location, and a composition that allows the implant to expand from the compressed form into an expanded form after the implant is inserted. The expanded form of the implant has a configuration that creates and maintains the desired anatomical space. The implant may also include a pharmaceutical element that treats disorders or diseases such as, but not limited to, inflammation and oncological conditions. The pharmaceutical element could be a drug eluding coating or a drug reservoir that permeates through a barrier to the treatment site.

Various delivery devices can be used to insert the present implants into the area being treated. The devices are adapted to retain the implant while the device is inserted into the desired location, and to controllably release the implant therein.

Due to the implant possibly being temporary, various retrieval devices can be used to remove the implant from the treatment site. Ideally the implant position, along with design features just superficial the dermis, allow retrieval and removal by a device requiring a small incision, and without the need of imaging equipment.

In an exemplary embodiment of the invention, a spacer for placement in an anatomical location includes a bladder being expandable from a deflated position to an inflated position, the bladder having at least one pair of opposing surfaces, the opposing surfaces being outer surfaces of the bladder and facing one another; a membrane extending between the opposing surfaces of the bladder; and an inflation port, the inflation port being in communication with the bladder and configured to receive inflation media to fill the bladder from the deflated position to the inflated position.

In another exemplary embodiment, the spacer includes wires defining an outer surface of the bladder. In yet another exemplary embodiment, the spacer includes at least one holder attached to the bladder, and the at least one holder is coupled to the wires.

In another exemplary embodiment, the wires are formed of nitinol. In yet another exemplary embodiment the inflation media is radiopaque. In yet another exemplary embodiment, the bladder is formed of polyethylene terephthalate. In yet another exemplary embodiment, the bladder is formed of a permeable material. In yet another exemplary embodiment, the inflation media comprises a medicinal substance.

In another exemplary embodiment, the bladder has a toroidal shape. In yet another exemplary embodiment, the membrane is formed of an additional bladder configured to elude medicine to the anatomical location.

In yet another exemplary embodiment, a spacer assembly for placement of a spacer in an anatomical position includes a spacer having a first holder and a second holder, the first holder being located proximally to the second holder, and a tension-compression mechanism having a pull tube and a push tube slidably coupled to one another. The pull tube is coupled to the second holder, and the push tube is coupled to the first holder.

In yet another exemplary embodiment, the pull tube is releasably coupled to the second holder. In yet another exemplary embodiment, the pull tube is located outside the push tube and includes at least one leg extending to the second holder. In yet another exemplary embodiment, the at least one leg is slidable in a direction of a longitudinal axis of the pull tube within a passage located in the first holder. In yet another exemplary embodiment, the pull tube includes a notch, and the pull tube is rotatable relative to the first holder about the longitudinal axis of the pull tube when the notch is located in the passage.

In another exemplary embodiment, a method for placement of an anatomical spacer includes placing a spacer inside a cannula. The spacer includes a bladder having at least one pair of opposing surfaces, an inflation port, the inflation port being in communication with the bladder and configured to receive an inflation media, and wires extending from a first holder to a second holder about an outer surface of the spacer. The method also includes retracting the cannula from the spacer at a predetermined site, with the inflation port located subcutaneously.

In yet another embodiment, the method further includes inflating the bladder with the inflation media from a deflated position to an inflated position. In yet another embodiment, the first holder and the second holder are located on opposite sides of the bladder and are attached thereto, and the method includes moving the first holder closer to the second holder. In yet another exemplary embodiment, a tension-compression mechanism is coupled to the first holder and the second holder, and the method includes actuating the tension-compression mechanism to position the spacer, and removing the tension-compression mechanism from the spacer.

In another embodiment of the invention, a spacer for placement in an anatomical location includes a helical spring having a toroidal shape; a pliable pouch covering the toroidal shape of the helical spring; and at least one holder coupled to the helical spring. In yet another embodiment, the helical spring is formed of nitinol.

### BRIEF DESCRIPTION OF THE DRAWINGS

FIG. 1 shows an isometric view of a toroidal shaped anatomical spacer in an expanded state according to aspects of the invention.

FIG. 2 shows an isometric view of a toroidal shaped anatomical spacer with opposing wire supports in an expanded state according to aspects of the invention.

FIGS. 3A and 3B show isometric views of an anatomical spacer with opposing wire supports in a folded (compressed) state according to aspects of the invention.

FIG. 4 shows an isometric view of the anatomical spacer with opposing wire supports in a partially deployed state according to aspects of the invention.

FIG. 5 shows an isometric view of a pliable pouch with opposing helical wire springs in the expanded state according to aspects of the invention.

### DETAILED DESCRIPTION OF THE INVENTION

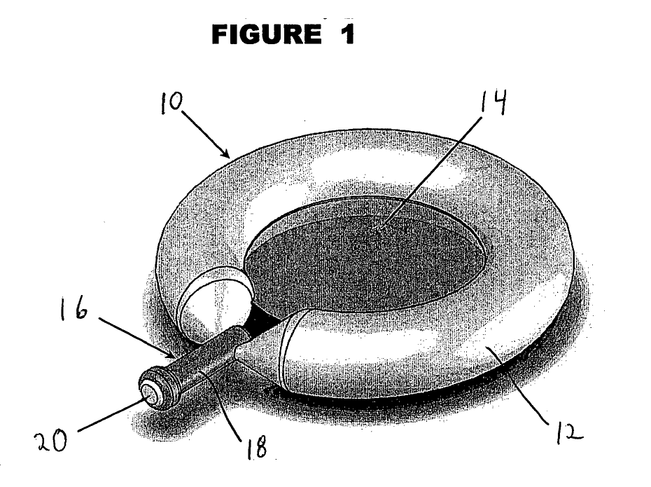

With reference to FIG. 1, a anatomical spacer 10, sometimes called an implant, can temporarily or permanently be implanted in or near a joint to prevent indirect or direct bone to tendon contact, also known as impingement. In one exemplary embodiment, the spacer 10 includes a bladder 12 having a toroidal shape, a membrane 14 tenting across the inner toroidal shaped space, and an inflation port 16 located at a junction of the bladder 12.

The bladder 12 is capable of pliable inflation, and could be made from a compressible, yet not stretchable (i.e., non-compliant) material such as polyethylene terephthalate, commonly known as PET. The bladder 12 may be folded similar to an angioplasty balloon, providing reduced profile for insertion down a cannula, as will be described below. An inflation media, formed from any fluid acceptable for implant applications, fills the bladder 12 to perform the function of the spacer, as described below. One possible inflation media is a radiopaque contrast medium to facilitate observation under x-ray imaging. In another embodiment, the bladder 12 is permeable and the inflation media is medicated, thus allowing the medicated inflation media to be released to an implant site to aid in reducing inflammation or other disorders.

The membrane 14 extends in the inner toroidal shaped space defined by the bladder 12, and is secured to an outer surface of the bladder 12. The membrane may be formed from a single sheet as shown in FIG. 1. On other embodiments, the membrane is formed from a flexible sock that encapsulates the bladder. The sock may have drug eluding qualities to provide medication to the implant site. The membrane itself may form an independent bladder, the independent bladder having its own dedicated port for filling. The independent bladder may act as a drug reservoir for dispensing of a pharmaceutical element to the implant site.

The inflation port 16 includes a manifold tube 18 and a septum 20. The port 16 is capable of receiving a needle for inflation of the bladder 12, as described below. The manifold tube 18 includes openings for communication of the inside of the manifold tube 18 with the inside of the bladder 12. The openings in the manifold tube 18 allow the inflation media to enter and inflate the bladder 12.

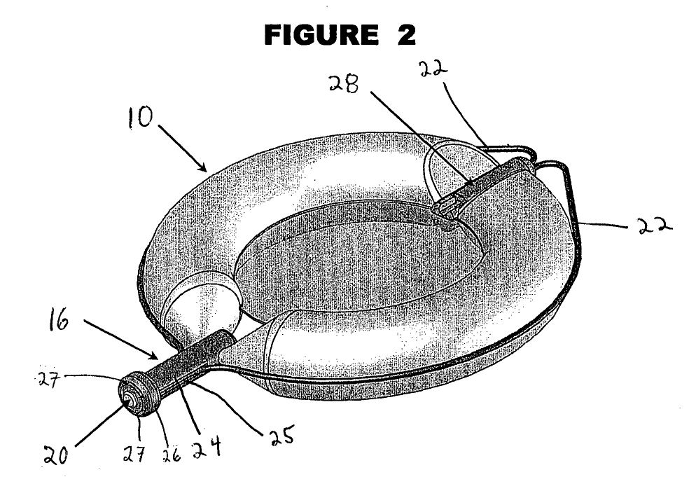

With reference to FIG. 2, a pair of opposing wire supports 22 extend around an outer circumference of the bladder 12 to give structural integrity to the implant 10. The port 16 includes a proximal holder 24 in which the wire supports 22 are held. The proximal holder 24 is secured to the outside of the manifold tube 18 (see FIG. 1), and includes slots 25 into which the opposing wires 22 are inserted and a rim 26 located around the septum. The rim 26 has portions removed which define passages 27. The passages 27 extend circumferentially around the manifold tube 18.

A distal holder 28 in which the wire supports are held is located diametrically opposed to the proximal holder 24. The distal holder 28 is secured to the bladder 12 and is oriented radially and in line with the port 16. The distal holder 28 is generally tube shaped, and the wire supports are grasped inside the tube shaped holder. The wire supports 22 extend from an outer end of the second holder 28 before curving back to the outer circumference of the bladder 12. The wire supports 22 may be fabricated from super elastic nitinol material to prevent permanent deformation when being compressed for insertion.

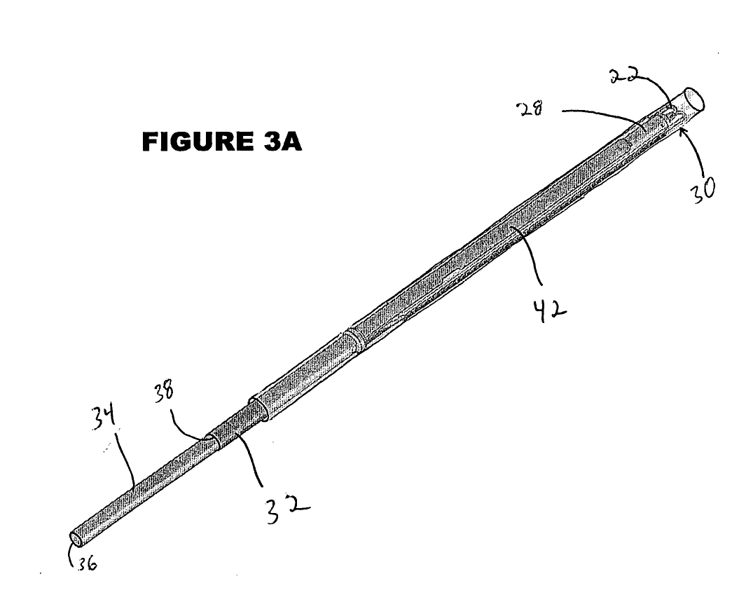

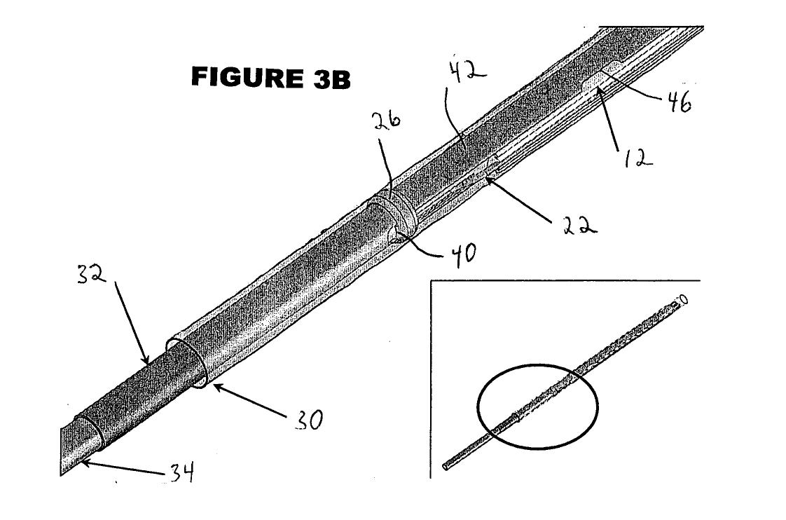

With reference to FIGS. 3A and 3B, the implant is capable of being compressed to a reduced profile for delivery to the implant site from a cannula 30. In order to fit into the cannula 30, the bladder 12 (see FIG. 1) may be folded similar to an angioplasty balloon, as is known in the art. The wire supports 22 are flexed to be able to fit into the delivery cannula 30. A tension-compression mechanism including a pull rod 32 and a push rod 34 is used to control the proximal holder 24 and the distal holder 28 and deploy the spacer 10 (see FIG. 1) at the implant site. The pull rod 32 and the push rod 34 are both generally tube shaped. The pull rod 32 is placed outside the push rod 34, with a proximal end 36 of the push rod 34 extending proximally from a proximal end 38 of the pull rod 32. A distal end 40 of the push rod 34 abuts at least one of a proximal end of the manifold tube 18 (see FIG. 1) and the rim 26.

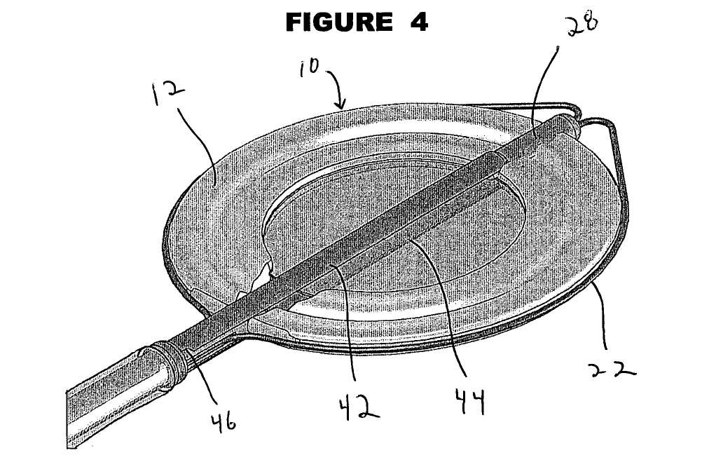

The pull rod 32 splits into two legs 42 and 44 (see also FIG. 4) that extend through the passages 27 (see FIG. 2) in the proximal holder 24. The legs 42 and 44 continue the curved profile of the pull rod 32 and include a notch 46 at a predetermined position, as described below. The legs 42 and 44 extend to the distal holder 28, and may be releasably coupled thereto.

The cannula 30 houses the spacer 10 (see FIGS. 1 and 2) during delivery to the implant site. Delivery of the spacer to the implant site is achieved by placement of the cannula 30 in the desired anatomical space by methods known in the art. The cannula 30 is then retracted as an operator holds the position of the proximal holder 24 and the distal holder 28 by holding the pull rod 32 and the push rod 44 in a stable position. The opposing wires 22 are deployed as the pull rod 32 is moved proximally over the stationary push rod 34, toward the proximal end 36 thereof. The distal end 40 of the push rod 34 bears against the proximal holder 24 and or the proximal end of the manifold tube 18 (see FIGS. 1 and 2), maintaining constant the position of the proximal holder 24, while the distal holder 28, coupled to the pull rod 32, moves proximally closer to the stationary proximal holder 24. The opposing wires 22 are also deployed as the push rod 34 is moved distally into the stationary pull rod 32. The second holder 28, coupled to the stationary pull rod 32, maintains a constant position as the push rod 34 bears against the proximal holder 24, moving it toward the stationary distal holder 28. The inflation port 16 and proximal holder 24, being located proximally, are the last items from the spacer 10 delivered out of the cannula 30.

With reference to FIG. 4, the opposing wires 22, which exert forces on surrounding objects to reach their natural curved state, can provide a displacement force needed to overcome possible obstructions during implantation, assuring that the spacer 10 can reach its intended deployed shape. The tension-compression mechanism can be manipulated to achieve proper positioning and remove anatomical obstructions, using relative movement between the pull rod 32 and the push rod 34, as described above. The notch 46 is located in the passages 27 (see FIG. 2), indicating that the wires 22 are in the natural state. The pull rod 32 can then be rotated along its longitudinal axis within the passages 27, releasing the coupling of the pull rod 32 and the distal holder 28.

The tension-compression mechanism can then be removed from the implant site as the pull rod 32 is pulled proximally from the passages 27 (see FIG. 2). Using this technique, the proximal holder 24 and the inflation port 16 can be positioned subcutaneously to allow superficial access and removal of the spacer 10.

The bladder 12, shown in a deflated state in FIG. 4, is then inflated to effectively create a spacer. The septum 20 is configured for needle penetration and delivery of the inflation media. The inflated bladder 12 (see FIG. 2) would prevent the bone from impinging against the sensitive portion of the tendon.

Removal of the spacer 10 may be accomplished by a small incision to access the port, needle puncture through the port septum 20, evacuation of the inflation media, and then complete removal of the deflated bladder.

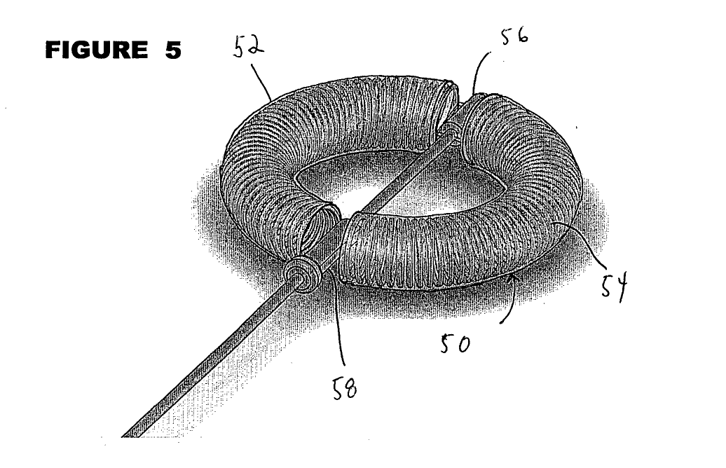

With reference to FIG. 5, an alternative embodiment spacer 50 includes a pliable pouch 52, two opposing helical spring members 54, a distal connector 56, and a proximal connector 58. The helical spring members 54 have an axis that follows a half circle, thus effectively creating a toroidal shape. Both of the spring members 54 are attached on each end to one of the connectors 56, 58. The opposing helical spring members 54 could be controlled by the distal connector 16 and proximal connector 17 being operated by a tension-compression mechanism, for example, as the tension-compression mechanism described above. The helical spring members could be fabricated from super elastic nitinol material to prevent permanent deformation when undergoing the extreme tension needed for delivery down a cannula.

After the implant is in proper position, the tension-compression mechanism would be released and removed. The nitinol helical springs could collapse on their sides when under high loading, yet the collapsed mode would still provide the spacer effect needed to prevent impingement. Removal of the implant may be accomplished by a small incision to retrieve the proximal connector, attachment to a removal tool (which could be same as delivery tool), activation of the tension-compression mechanism to obtain a small profile, and then complete removal of the implant.

The implant retrieval and removal procedure for all the embodiments might not require imaging equipment, and could possible be performed in a physician office setting. Although the embodiments above refer to a toroidal or “donut shaped” spacer, other shapes that better approximate the particular anatomy being treated are conceivable.

While the invention has been described in its preferred embodiments, it is to be understood that the words which have been used are words of description and not of limitation. Therefore, changes may be made within the scope of the appended claims without departing from the true spirit and scope of the invention.

What is claimed is:

1. A spacer for placement in an anatomical location, the spacer comprising:
a bladder being expandable from a deflated position to an inflated position, the bladder having at least one pair of opposing surfaces, the opposing surfaces being outer surfaces of the bladder and facing one another;
a membrane extending between the opposing surfaces of the bladder; and
an inflation port, the inflation port being in communication with the bladder and configured to receive inflation media to fill the bladder from the deflated position to the inflated position.

2. The spacer according to claim 1 and further comprising wires defining an outer surface of the bladder.

3. The spacer according to claim 2 and further comprising at least one holder attached to the bladder, wherein the at least one holder is coupled to the wires.

4. The spacer according to claim 3 wherein the wires are formed of nitinol.

5. The spacer according to claim 1 wherein the inflation media is radiopaque.

6. The spacer according to claim 1 wherein the bladder is formed of polyethylene terephthalate.

7. The spacer according to claim 1 wherein the bladder is formed of a permeable material.

8. The spacer according to claim 7 wherein the inflation media comprises a medicinal substance.

9. The spacer according to claim 1, wherein the bladder has a toroidal shape.

10. The spacer according to claim 1, wherein the membrane comprises an additional bladder configured to elude medicine to the anatomical location.

11. A spacer assembly for placement of a spacer in an anatomical position, the spacer assembly comprising:
a spacer comprising a first holder and a second holder, the first holder being located proximally to the second holder; and
a tension-compression mechanism comprising a pull tube and a push tube slidably coupled to one another;
wherein the pull tube is coupled to the second holder, and wherein the push tube is coupled to the first holder.

12. The spacer assembly according to claim 11, wherein the pull tube is releasably coupled to the second holder.

13. The spacer assembly according to claim 12, wherein the pull tube is located outside the push tube and includes at least one leg extending to the second holder.

14. The spacer assembly according to claim 13, wherein the at least one leg is slidable in a direction of a longitudinal axis of the pull tube within a passage located in the first holder.

15. The spacer assembly according to claim 14, wherein the pull tube includes a notch, wherein the pull tube is rotatable relative to the first holder about the longitudinal axis of the pull tube when the notch is located in the passage.

16. A method for placement of an anatomical spacer, the method comprising:
placing a spacer inside a cannula, the spacer comprising:
a bladder having at least one pair of opposing surfaces,
an inflation port, the inflation port being in communication with the bladder and configured to receive an inflation media, and
wires extending from a first holder to a second holder about an outer surface of the spacer; and
retracting the cannula from the spacer at a predetermined site, wherein the inflation port is located subcutaneously.

17. The method according to claim 16 and further comprising inflating the bladder with the inflation media from a deflated position to an inflated position.

18. The method according to claim 17, wherein the first holder and the second holder are located on opposite sides of the bladder and attached thereto, and further comprising moving the first holder closer to the second holder.

19. The method of claim 18, wherein a tension-compression mechanism is coupled to the first holder and the second holder, and further comprising actuating the tension-compression mechanism to position the spacer, and removing the tension-compression mechanism from the spacer.

20. A spacer for placement in an anatomical location, the spacer comprising:
a helical spring having a toroidal shape;
a pliable pouch covering the toroidal shape of the helical spring; and
at least one holder coupled to the helical spring.

21. The spacer according to claim 20, wherein the helical spring is formed of nitinol.
